(19) United States
(12) Patent Application Publication
(10) Pub. No.: US 2009/0208559 A1
(43) Pub. Date: Aug. 20, 2009

(54) DEVICE FOR DELIVERING ACTIVE INGREDIENTS TO HUMANS, ANIMALS AND PLANTS

(76) Inventors: Bruce Drew, Essex (GB); Shilpan Pravinchandra Patel, Maharashtra (IN); Nikita Kamlesh Thanawala, Maharashtra (IN)

Correspondence Address: AMIN Talati, LLC, 444 NORTH ORLEANS STREET, SUITE 400, CHICAGO, IL, 60654, US

(21) Appl. No.: 12/304,850

(22) PCT Filed: Jun. 14, 2007
(86) PCT No.: PCT/IN07/00240

§ 371 Date: Mar. 27, 2009

(30) Foreign Application Priority Data
Jun. 14, 2006 (IN) 1569/MUM/2005

(51) Int. Cl. A61K 9/70; A61P 43/00

(52) U.S. Cl. 424/448; 424/443; 424/447

(57) ABSTRACT

A device for controlled delivery of an active ingredient to a receiving site (1) such as a plant, human or animal. The device comprises a filmic substrate including a water soluble carrier layer (2) disposed between first and second water soluble control layers (3, 4). The second control layer (4) of the filmic substrate is provided with adhesive (5) on the side remote from the carrier layer (2) for attaching the device to the receiving site (1) with the first control layer (3) outermost. The first and second control layers (3, 4) having perforated regions (9, 8) whereby the carrier layer (2) can be dissolved by wetting the first control layer (3) with an aqueous medium that passes through the perforations (9) of the first control layer (3) releasing the active ingredient into solution that passes through the perforations (8) of the second layer (4) to come into contact with the receiving site (1).

### FIELD OF INVENTION

This invention relates to a device for delivery of one or more active ingredients to a receiving site. More especially, the invention relates to a multilayered device that can be applied to one or more parts of plants, humans or animals externally to provide sustained release of active ingredients such as nicotine, drugs; neutraceutical active ingredients such as vitamins, biological active ingredients such as cell growth inhibitors or probators; pesticidal active ingredients such as herbicides, fungicides, insecticides, molluscicides; plant growth regulators, hormones, plant nutrition agents such as foliar fertilisers; biocides, disinfectants and the like. The invention also relates to a method for delivery of one or more active ingredients to a receiving site.

### BACKGROUND OF INVENTION

It is well known that active ingredients such as nicotine can be delivered through pores of the skin or wounds can be healed by delivering active drugs through a cut in the skin due to the action of blood circulation or the growth of a plant can be enhanced by externally delivering certain hormones.

The delivery of such active ingredients is dependent upon the transfer rate between the device containing the active ingredient, which shall be called the ‘donating device’ for the purpose of this invention and the ‘receiving site’ i.e. the surface of a human, animal or plant body.

WO 2006/067514 describes a dosing system employing water-soluble or water dispersible products containing active ingredients embedded within and/or coated upon a water-soluble film which are released at a prescribed rate into an aqueous environment through one or more perforated less readily soluble water-soluble films. The dosing system described in this application can be used for delivering pharmaceutical active ingredients externally or internally to a human or animal body, and can be embodied in the form of a patch that is applied to a site to be treated via a water soluble adhering layer that adheres to the site with the ingredient(s) provided by a carrier layer sandwiched between a perforated layer for delivery of the ingredients to the site and a non-perforated protecting layer.

The adhering layer, i.e. a readily dissolving WSF, adheres on the surface due to the presence of an aqueous medium at the site to be treated, whether this moisture is present due to bodily or other fluids or has deliberately been applied to the surface for the application of the product. The moisture of aqueous medium present on the surface then passes through the perforated layer and causes dispersion/dissolution of the carrier WSF layer containing active ingredients such as certain drugs. Thus, the active ingredients will pass through the perforated layer and are delivered to the desired site.

The multilayered films of WO 2006/067514 can be used for delivering active ingredients to human beings or animals either internally or externally. However, when applying such a multilayered film containing an active embedded film is applied externally e.g. a wound, there are chances that the body fluid or the aqueous medium applied at the site is not sufficient to dissolve the adhering layer as well as the carrier WSF layer incorporated or coated with active ingredients.

Hence, there is a need for improving this product for external application of the same.

### DEFINITIONS

As used herein the terms “cut plant”, “animal or human body”, “device” and “wetting” as they are used in the description of this invention and in the claims are defined as follows:

The term “cut plant” means any plant that is cut for subsequent sale or re-use including therefore flowers, ornamentals and shrubs, certain vegetables such as lettuce, and certain fruit such as bananas, pineapples, although these examples are by no means limiting.

The term “device” means the whole construction of the product which is derived by using the process as mentioned in this invention. This includes, but is not limited to, a multi-layered water soluble film with one or more of them being perforated so as to create a time delay system by selectively and discreetly wetting the said construction of films.

The term “wetting” means wetting of the “device” from the exterior perforated layer with aqueous medium.

The water soluble materials described and claimed herein require the presence of water to dissolve/disperse the material. In contrast, biodegradable materials are materials that are capable of being broken down safely by living microorganisms, principally bacteria and fungi, into innocuous products (e.g. the raw materials of nature) and disappear into the environment. This is a totally different process and the presence of water is not always necessary for a material to biodegrade. The present invention excludes materials that are biodegradable.

### SUMMARY

The present invention seeks to provide an improved dosing system for external delivery of active ingredients to plants, humans or animals and has as a preferred aim the development of a better delivery system for ease of dissolution of the water soluble film layer incorporated with active ingredients for sustained release of the active ingredients.

According to one aspect, the invention provides a multilayered device comprising a first layer of at least partially perforated water soluble film; a second layer of a water soluble film incorporated with at least one active ingredient; a third layer of at least partially perforated water soluble film; and a fourth layer of an adhesive for securing the device to a receiving site.

By this invention, the water soluble film incorporating at least one active ingredient is positioned between two at least partially perforated water soluble films such that, when the device is attached to a receiving site by means of the adhesive, the device has an outer surface provided by one of the perforated water soluble films that allows an aqueous medium to penetrate the device through the outer surface and come into contact with the water soluble film incorporating the active ingredient(s) thereby causing the film to dissolve releasing the active ingredient(s) which then pass in solution through the other perforated layer to come into contact with the receiving site.

In this way, the active ingredient(s) can be delivered to a receiving site over an extended period of time by applying a prescribed amount of the aqueous medium to the external perforated area of the outer surface of the device at regular intervals as prescribed by the supervisor. The external perforated area acts as a regulating substrate allowing only an exact dose of the aqueous medium to pass through. The amount of aqueous medium that passes through the perforations can be varied by adjusting the number and/or size of perforations per unit area. Once the aqueous medium has passed through the perforations, it begins to dissolve the internal water-soluble film layer incorporating the active ingredient(s), thereby releasing a measured dose of active ingredient(s) into solution and hence available for use at the receiving site. This process can be repeated and controlled by subsequent application of the prescribed aqueous medium.

It has been found that the invented water-soluble device may be used to heal wounds, for controlled delivery of active ingredients to humans or animals or to heal damaged plants in an economical manner as when needed by rewetting the device with the aqueous medium, for example water, which results in corresponding saving in time and money.

Preferably, the active ingredients include but are not limited to nicotine, pharmaceutical active ingredients, often known as drugs; neutraceutical active ingredients such as vitamins, biological active ingredients such as hormones, cell growth inhibitors or probators; pesticidal active ingredients such as herbicides, fungicides, insecticides, molluscicides; plant growth regulators, hormones, plant nutrition agents such as foliar fertilisers; biocides, disinfectants although this list is by no means limiting.

The water soluble films of the device may be formed of edible raw materials or non-edible raw materials. The device may comprise edible raw materials only or non-edible raw materials only or a combination of edible and non-edible raw materials.

Preferred edible raw materials include, but are not limited to polyethylene glycol, glycerin, propylene glycol, polyvinylpyrrolidone, proteinaceous binders such as gelatin, modified gelatins such as phthaloyl gelatin, sodium alginate, polysaccharides such as starch, gum Arabic, pullulan and dextrin, tragacanth gum, guar gum, acacia gum, amylose, sweeteners, pectin, chitin, chitosan, levan, elsinan, collagen, zein, gluten, soy protein isolate, casein, shellac, water-soluble cellulose derivatives and the like. The cellulose derivatives used are methyl cellulose, hydroxy propyl cellulose, hydroxy propyl methyl cellulose, hydroxy propyl ethyl cellulose, hydroxy ethyl cellulose, carboxy methyl cellulose. However, these raw materials are by no means limiting.

Preferred non edible raw materials include, but are not limited to, polyvinyl alcohol copolymer ionomers, polyvinyl alcohol homopolymer, non-ionomeric poly vinyl alcohol polymer, polymethacrylate, polyvinyl alchohol, polyacrylamide, polymethacrylamide, polyacrylic acid, polymethacrylic acid, polyurethane. However, these raw materials are by no means limiting.

The device may be applied to the receiving site in any suitable form including, but not limited to, a bandage, wound dressing, patch, tape or label which will deliver the active ingredient(s) to plants, humans or animals. For application to plants, the device preferably provides mechanical support to the plant during the healing process. The degree of mechanical support may be varied by adjusting the formulation, thickness and elasticity of the filmic substrate.

Preferably, the device can be made using film and coatings in a fashion which makes a relatively transparent or translucent product, whereby the user can observe the healing process and re-wet the device to release active ingredients in a timely manner.

Preferably, the adhesive layer is provided at selective places, e.g. only around the edges of the device such that a center part of the device is exposed to the plants or human body or animal body to be treated. In this way, active ingredient(s) released by aqueous medium penetrating the outer surface of the device can come into contact with the receiving site within the center part of the device

In the case of delivery of active ingredients like, but not limited to, nicotine or pain killers or such other drugs, the device can be wetted externally and intermittently applying an aqueous medium, for example water, to re-wet the device so as to deliver the active ingredient(s) to comfort and cure the patient or animal or plant.

Preferably, the active ingredient(s) assist the healing process. Preferably, the active ingredient(s) protect the target area during the healing process.

The water soluble films may be formulated to be fully soluble in water of a pre-determined temperature range, whether in cold water i.e. ≧4° C., in warm water i.e. ≧40° C., or in hot water ≧60° C. As will be understood, films that are soluble in cold water, i.e. water having a temperature greater than or equal to 4° C., will also be soluble in warm water and hot water, and films that are soluble in warm water, i.e. water having a temperature greater than or equal to 40° C., will also be soluble in hot water but insoluble in cold water, while films that are soluble in hot water, i.e. water having a temperature greater than or equal to 60° C., will be insoluble in cold water and warm water. In this way, we can arrange for the carrier layer to dissolve before the control layers by appropriate selection of the solubility of the water soluble materials employed for the carrier layer and control layers.

Preferably, the water soluble film of the layer incorporating the active ingredient(s) is soluble in water having a lower temperature than the water soluble film of the other layers. For example, the water soluble film of the layer incorporating the active ingredient(s) may be soluble in cold water with the water soluble film of the other layers being soluble in warm or hot water. In this way the water soluble film incorporating the active ingredient(s) is preferentially dissolved to release the active ingredient(s) by wetting the device with cold water.

Preferably, the adhesive layer is provided with a release liner that is removable when it is desired to attach the device to a receiving site. The adhesive may be of any suitable type and is preferably a pressure sensitive adhesive.

The adhesive layer may comprise a water soluble adhesive that is soluble in water at a temperature higher than the water soluble film incorporating the active ingredient(s) and is preferably soluble in water at the same temperature as the perforated water soluble films. In this way, when the treatment is completed, the device may be removed and thrown away. Alternatively, the device may be dissolved in situ by wetting with water of the appropriate temperature.

In another embodiment of the invention, particularly suitable for application of the device to plants, it is intended that the device will not require to be subsequently removed from the plant as, over a period of time, it will either be absorbed into the plant without detrimental effects, or will be dissolved by the combined effects of rain and dew. The time taken for the device to dissolve may be varied by selecting an appropriate grade of filmic substrate and an appropriate formulation of adhesive. It will be appreciated that the selection of suitable materials will be influenced by the weather conditions to which the plant is exposed.

In another embodiment of the present invention, suitable for application of the device to humans, animals or plants, the outer layer which is perforated to accept the water for rewetting the device and/or the inner layer which is perforated to deliver the solution containing the active ingredient(s) to the receiving site may be made of materials which dissolve in warm or hot water, so as to for example, to allow the device to be exposed to normal rains or perspiration during the process of healing, and the layer incorporating the active ingredient(s) may be cold water soluble to ensure that the perforated layers which deliver the cold water to activate the device and deliver the active ingredient(s) to the receiving site are not dissolved during the desired healing process.

In yet another embodiment of the present invention, the device can be conveniently washed off after the desired results of curing or active ingredient delivery is completed, for example by using a continuous flow of water having a temperature which is slightly above the dissolving temperature range of the device.

For application to plants, the device may be designed to incorporate active ingredients known to assist in the healing process whether directly as in the case of fimgicides, bactericides, mouldicides, disinfectants, biocides, plant stimulants, plant hormones which help in growth of plants, micro-nutrients, and foliar fertilisers, or indirectly as in the case of insecticides which protect the plant during the healing process from unwanted insect attack, molluscicides which protect the plant during the healing process from unwanted mollusic attack, and plant growth regulators.

For application to humans or animals, the device may be designed to incorporate active ingredients known to assist in the healing process whether directly as in the case of vitamins, drug regulators, cell growth inhibitors cell growth regulators, pain killers, nicotine, nutrients etc.

These active ingredients for plants, humans and animals are provided by way of example only and it will be understood that other active ingredients can be used for the purpose of this invention and we do not intend to be limited to the examples provided.

One or more active ingredients of any of the above types may be incorporated individually or in combination into the water soluble film of the layer disposed between the perforated layers. In one embodiment, one or more active ingredients may additionally be incorporated into the adhesive layer for transfer to the receiving site. The active ingredients incorporated into the water soluble film and the adhesive may be the same or different. For example, the water soluble film may incorporate one or more active ingredients to assist the healing process and the adhesive may incorporate on or more active ingredients for pain relief. All combinations of active ingredients are included within the scope of the invention.

It will, of course, be seen that if one or more active ingredients are incorporated into the adhesive layer which is then placed in direct contact with the receiving site, the device can deliver active ingredients in a similar way that a gel/balm delivers small doses of a pain killer through the skin of the human body over a pre-determined period of time. The benefits of this will be greater in the case where the active ingredients are systemic in nature and will therefore be subsequently transported throughout the plant or a human or animal body. In cases where rapid uptake of active ingredient is required, external application of water to preferentially wet the bandage shall release the actives for delivery as needed.

In one application the invention is therefore able to provide, if both are required, a self-adhesive device for repairing a damaged plant from a mechanical standpoint and a novel delivery system for dispensing active ingredients to the plant, whether to help to heal the site of the wound, splice or graft, or for other reasons. It will be noticed that the delivery of active ingredients to the plant in this way is particularly safe and efficacious as the active ingredients are placed in intimate contact with the tissue of the plant without contamination of the environment surrounding the plant or the user.

In a further application of the invention, the stems of cut plants or cuttings are bound, either individually or in bunches, with a self-adhesive device incorporating one or more active ingredients in order to prolong their life after cutting, firstly in transport from the grower to the consumer and secondly after being placed in water by the consumer.

Similarly, the post harvest life of fresh produce may be prolonged by protecting its cut surfaces, particularly against fungal attack, by means of a self-adhesive device according to the invention placed over the cut surface as quickly as possible after harvesting.

Examples of such fresh produce are certain vegetables such as lettuce and certain fruit such as bananas and pineapples although these examples are by no means limiting.

It will be evident that one or both of the filmic substrate and the adhesive can be coloured within their mass and the filmic substrate may be printed, in order to camouflage the presence of the device on the plant and to enable marketing and technical information to be provided to the user.

Preferably, the layer incorporated with active ingredients is coloured. Hence, when the layer starts dissolving, the colour of the film also starts dispersing and a person can observe whether the active ingredient is being delivered to the wound or not and when the patch is almost colourless, it can be removed.

In yet another application of the present invention, which is not related to a healing process, one or more herbicides are incorporated into the device, preferably into the formulation of the adhesive, for application to one or more leaves or stems of the plant. In this way, the herbicide or combination of herbicides is absorbed into the plant and unwanted plants can thereby be eliminated.

As will now be understood, in some embodiments, the present invention provides a method of applying active ingredients to plants in which the active ingredient(s) are transmitted safely and efficaciously to the plant without contaminating the user, the environment, neighbouring plants or crops, and the self-adhesive tape or label will over time dissolve, become absorbed by the plant, or disperse either whilst still attached to the plant or in a subsequent composting process.

According to another aspect, the invention provides a device for controlled delivery of one or more active ingredient(s) to a receiving site, the device comprising a filmic substrate including a water soluble carrier layer disposed between first and second water soluble control layers, the second control layer of the filmic substrate being provided with adhesive on the side remote from the carrier layer for attaching the device to a receiving site with the first control layer outermost, and the first and second control layers having perforated regions whereby the carrier layer can be dissolved by wetting the first control layer with an aqueous medium that passes through the perforations of the first control layer releasing the active ingredient into solution that passes through the perforations of the second layer to come into contact with the receiving site.

According to yet another aspect, the invention provides a method of delivering an active ingredient to a receiving site, the method including the steps of providing a self-adhesive device comprising a filmic substrate having a water soluble carrier film incorporating at least one active ingredient disposed between first and second water soluble control films wherein the first and second films are at least partially perforated and the second film is provided with an adhesive layer, attaching the device to a receiving site with adhesive layer, and wetting the first control layer of the device with an aqueous medium whereby the aqueous medium passes through the perforations to dissolve gradually the carrier layer to release the active ingredient into solution to pass through the perforations of the second control layer to come into contact with the receiving site.

Preferably, the method includes periodically re-wetting the first control layer.

Preferably, the method includes providing perforations in the second control layer that are larger than the perforations in the first control layer.

The invention will now be described in more detail, by way of the following examples for the production of multi-layer devices with reference to the accompanying drawings.

### BRIEF DESCRIPTION OF THE DRAWINGS

FIG. 1 shows a multilayer device according to the invention.

### DETAILED DESCRIPTION OF PREFERRED EMBODIMENTS

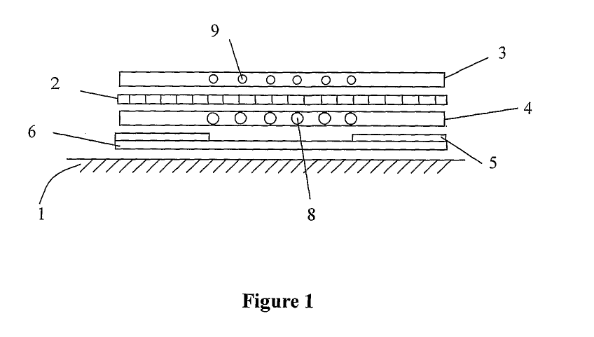

In FIG. 1 reference number 1 depicts a receiving site such as the surface of a plant or the skin of a human or animal to which a multilayered device according to the invention is to be applied for delivery of at least one active ingredient to the site 1 in a controlled manner.

The device includes a carrier layer 2 comprising a water soluble film containing one or more active ingredients disposed between two control layers 3, 4 each comprising a water soluble film. The water soluble films of the control layers 3, 4 are formulated to dissolve in water at a higher temperature than the water soluble film of the carrier layer 2. For example, the carrier layer 2 may dissolve in cold water (≧4° C.) with the control layers 3, 4 dissolving in warm water (≧40° C.) or hot water (≧60° C.). The control layers 3, 4 and carrier layer 2 may be made of edible or non-edible raw materials.

The said water soluble film incorporated with active ingredients can be prepared by mixing precise quantities of active, ingredients into the known water soluble film formulation; casting the said known formulation onto a liner or conveyor and drying the same.

Further, the said water soluble film layer incorporated with active ingredients can also be prepared by spraying/sprinkling of the active ingredients onto a water soluble film prior to its drying.

Further, precise quantities of active ingredients can be dispensed in between two pre formed water soluble films to form a layer having active ingredients entrapped in between two water soluble films.

Optionally, active ingredients can be coated on the surface of the carrier layer 2.

The coating or sprinkling of active ingredients can be done intermittently or fully or two active ingredients can be sprinkled or coated alternatively if desired.

The layers 2, 3, 4 are laminated together to produce a filmic substrate in which the control layer 3 is intended to provide the outer surface of the device in use and the control layer 4 is provided with an adhesive layer 5 for securing the device to the receiving site. The adhesive layer 5 is covered by a removable release liner 6 to protect the adhesive until it is required to attach the device to the receiving site 1. As shown the adhesive layer 5 does not cover all of the control layer 4 and is applied to the marginal edge regions only of the control layer 4 leaving a central area of the control layer 4 free of adhesive.

The central area of the control layer 4 is formed with a series of perforations 8. The number and size of the perforations 8 per unit area is chosen to control the rate of delivery of the active ingredients to the receiving site through the perforations 8. A central area of the control layer 3 aligned with the central area of the control layer 4 is also provided with a series of perforations 9. The number and size of the perforations 9 per unit area is chosen to control the rate of delivery of aqueous medium such as water to the carrier layer 2 through the perforations 9.

Two or more layers can be laminated by any suitable process. For example by applying water to the perforated water soluble film which, being, hydrophilic in nature, becomes a little tacky so that, when another water soluble film comes in contact with the perforated layer, it will adhere to the perforated layer. Alternatively, the layers can be laminated by a heat lamination process.

The device may take any suitable form for application to a plant, human or animal. For example the device may comprise a bandage or wound dressing or patch or tape or label.

In use, the device is attached to the receiving site 1 to position the central perforated area of the device over the region to be treated. The device is then wetted by applying aqueous medium such as water externally to the perforated area of the outer layer 3. The aqueous medium passes through the perforations 9 of the outer control layer 3 in a controlled manner to dissolve gradually the carrier layer 2 to release the active ingredient(s) into solution which then pass through the perforations 8 of the inner control layer 4 in a controlled manner to come into contact with the region to be treated where the active ingredient(s).

The number and/or size of the perforations 8 in the inner control layer 4 is preferably greater than that of the perforations 9 in the outer control layer 3 so that the solution containing the dissolved active ingredient(s) preferentially passes through the inner control layer 4 to contact the region to be treated. The adhesive layer 5 is preferably a pressure sensitive adhesive and is also water soluble typically at a temperature higher than the carrier layer 2. On completion of the treatment, the device can be removed from the site or dissolved by applying water of the appropriate temperature or left in situ to gradually dissolve over time.

### Example 1

a. A hot water soluble film batch formulation comprising 18-20% fully hydrolyzed polyvinyl alcohol, wherein the degree of hydrolysis of polyvinyl alcohol ranges from 91% to 99.9%; and 2-5% of a plasticizer such as glycerol was cast on a detachable liner and dried to form a hot water soluble film having a thickness of 60 microns.
b. The hot water soluble film was partially perforated by a dye cutting process so that the perforations were present only in the central part of the film. The size of the perforations was 1.7 mm diameter. This film formed the perforated hot water soluble control layer (3) described above
c. Another hot water soluble film having the same formulation as in step ‘a’ was cast and dried so as to form a hot water soluble film having thickness of 60 microns.
d. The hot water soluble film as formed in step ‘c’ was partially perforated by a dye cutting process so that the perforations were present only in the central part of the film. The size of the perforations was 2.5 mm diameter. This film formed the perforated hot water soluble control layer (4) described above.
e. A cold water soluble film batch formulation comprising 15-20% pullulan, 2-5% plasticizer and 6-7% of auxin (a hormone for plant growth) was prepared and cast on a detachable liner so as to form a cold water soluble film incorporated with auxin having a thickness of 25 microns and the detachable liner delaminated. This film formed the cold water soluble carrier layer (2) described above.
f. The three layers described above were laminated so that the carrier layer (2) of cold water soluble film was sandwiched in between the two perforated control layers (3), (4) of hot water soluble film. The lamination was carried out by a heat lamination process.
g. A water based pressure sensitive adhesive (PSA) was coated on a release liner only at the edges and dried to form the adhesive layer (5) with release liner (6) described above. The adhesive side of the release liner was combined with the control layer (4) of the perforated hot water soluble film having the larger perforations to align the perforations with the adhesive free zone of the release liner to form the device described above.
h. The laminate along with the adhesive and release liner was cut into a patch of size 10 cm×10 cm so as to have 2 mg of Auxin in this patch.
i. The patch as formed in step 8 was applied to the stem of a plant after delaminating the detachable liner and 10 ml of water was sprayed on to the patch at regular intervals of time so as to release auxin in a controlled manner.

### Example 2

a. A hot water soluble batch film formulation comprising 18-20% fully hydrolyzed polyvinyl alcohol, wherein the degree of hydrolysis of polyvinyl alcohol ranges from 91% to 99.9%; and 2-5% of a plasticizer such as glycerol was cast on a detachable liner and dried so as to form a transparent hot water soluble film having a thickness of 40 microns.
b. The hot water soluble film was partially perforated by a dye cutting process so that the perforations were present only in the central part of the film. The size of the perforations was 1.5 mm diameter. This film formed the perforated hot water soluble control layer (3) described above.
c. Another hot water soluble film having the same formulation as in step ‘a’ was cast and dried so as to form a transparent hot water soluble film having thickness of 60 microns.
d. The said hot water soluble film as formed in step ‘c’ was partially perforated by a dye cutting process so that the perforations were present only in the central part of the film. The size of the perforations was 2.5 mm diameter. This film formed the perforated hot water soluble control layer (4) described above.
e. A cold water soluble film batch formulation comprising 15-20% pullulan, 2-5% plasticizer, 10-11% of neomycin and 1-2% of a red colour dye was prepared and cast on a detachable liner so as to form a pink coloured cold water soluble film incorporated with neomycin having a thickness of 35 microns and the detachable liner delaminated. This film formed the carrier layer (2) described above.
f. The three layers as described above were laminated so that the carrier layer (2) of cold water soluble film was sandwiched in between the two perforated control layers (3), (4) of hot water soluble film. The lamination was carried out by a heat lamination process.
g. A water based pressure sensitive adhesive (PSA) incorporated with 10-11% of methyl salicylate which will act as a pain killer was coated on a release liner only at the edges and dried to form the adhesive layer (5) and release liner (6) described above. The adhesive side of the release liner was combined with the control layer (4) of the perforated hot water soluble film having larger perforations to align the perforations with the adhesive free zone of the release liner to form the device described above.
h. The laminate along with the adhesive and release liner was cut into a patch of size 5 cm×8 cm.
i. The patch as formed in step ‘h’ was applied on a wound of human being after delaminating the detachable liner and some amount of water was sprayed on to the patch at regular intervals so as to release neomycin in a controlled manner. The patch will also relieve the pain in the surrounding areas of the wound by release of the pain killer present in the adhesive which will act as a balm. Also, as the water is sprayed on the patch, the carrier layer (2) containing neomycin starts dissolving and the pink colour of the film start dispersing. In this way, a person can observe whether the active ingredient is being delivered to the wound or not and when the patch is almost colourless, it can be removed.

While the invention has been described using films that are perforated, it will be understood that the invention includes films that allow an aqueous medium to pass through the film together with any active ingredient where present; and the terms perforation and perforated are to be construed accordingly. It will also be understood that the invention has application to water soluble films that are completely soluble in water having a pre-determined temperature to form a homogeneous solution and to water soluble films that are partially soluble in water having a pre-determined temperature to form a temporary or permanent suspension. For example, when removing the device after use it may not be necessary for the device to be completely water soluble in order to disperse the device from the receiving site. Thus, the carrier film may be completely water soluble with the control films and adhesive layer being partially soluble.

The invention is not limited to the examples described herein and all applications and uses of the invented device are included within the scope of the invention as defined in the following claims.

We claim:

1. A multilayered device comprising:
a first layer of at least partially perforated water soluble film;
a second layer of a water soluble film incorporated with at least one active ingredient;
a third layer of at least partially perforated water soluble film; and
a fourth layer of an adhesive for securing the device to a receiving site.

2. A device according to claim 1 wherein, the active ingredients are selected from a group comprising pharmaceutical active ingredients, neutraceutical active ingredients, nicotine, biological active ingredients, cell growth inhibitors or probators, pesticidal active ingredients, fungicides, insecticides, molluscicides, plant growth regulators, hormones, plant nutrition agents, biocides, and disinfectants.

3. A device according to claim 1 wherein the device is formed of edible raw materials or non-edible raw materials or a combination of edible and non-edible raw materials.

4. A device according to claim 3 wherein edible raw materials are selected from a group comprising polyethylene glycol, glycerin, propylene glycol, polyvinylpyrrolidone, proteinaceous binders such as gelatin, modified gelatins such as phthaloyl gelatin, sodium alginate, polysaccharides such as starch, gum Arabic, pullulan and dextrin, tragacanth gum, guar gum, acacia gum, amylose, sweeteners, pectin, chitin, chitosan, levan, elsinan, collagen, zein, gluten, soy protein isolate, casein, shellac, and water-soluble cellulose derivatives.

5. A device according to claim 4 wherein cellulose derivatives are selected from a group comprising methyl cellulose, hydroxy propyl cellulose, hydroxy propyl methyl cellulose, hydroxy propyl ethyl cellulose, and carboxy methyl cellulose.

6. A device according to claim 3 wherein non-edible raw materials are selected from a group comprising polyvinyl alcohol copolymer ionomers, polyvinyl alcohol homopolymers, non-ionomeric poly vinyl alcohol polymers, polymethacrylate, polyvinyl alcohol, polyacrylamide, polymethacrylamide, polyacrylic acid, polymethacrylic acid, and polyurethane.

7. A device according to claim 1 in a form of a bandage, wound dressing, patch, tape or label.

8. A device according to claim 1 wherein the device provides mechanical support to a plant during a healing process and the mechanical support is adjustable by varying one or more of a formulation, thickness and elasticity of one or more of the water soluble films.

9. (canceled)

10. A device according to claim 1 wherein the device is transparent or translucent.

11. A device according to claim 1 wherein the adhesive layer is provided on non-perforated regions of the third layer.

12. (canceled)

13. A device according to claim 11 wherein a perforated region of the first layer is aligned with a perforated region of the third layer and the perforations of the first layer are smaller than the perforations of the third layer.

14. (canceled)

15. A device according to claim 1 wherein the water soluble film of the second layer incorporating the active ingredient(s) is soluble in water having a lower temperature than the water soluble film of the first and third layers.

16. (canceled)

17. A device according to claim 1 wherein the adhesive layer is provided with a release liner that is removable prior to attaching the device to a receiving site.

18. A device according to claim 1 wherein the adhesive is a pressure sensitive adhesive.

19. A device according to claim 1 wherein the adhesive comprises a water soluble adhesive that is soluble in water at a temperature higher than the water soluble film of the second layer incorporating the active ingredient(s).

20. A device according to claim 19 wherein the adhesive is soluble in water at the same temperature as the perforated water soluble films of the first and third layers.

21. A device according to claim 1 wherein the adhesive layer incorporates at least one active ingredient the same or different to that of the second layer.

22. (canceled)

23. A method of delivering an active ingredient to a receiving site, the method including the steps of:
providing a self-adhesive device comprising a filmic substrate having a water soluble carrier film incorporated with at least one active ingredient disposed between first and second water soluble control films, wherein the first and second water soluble control films are at least partially perforated and the second water soluble control film is provided with an adhesive layer;
attaching the device to a receiving site with an adhesive layer; and
wetting the first water soluble control film of the device with an aqueous medium whereby the aqueous medium passes through the perforations to dissolve gradually the water soluble carrier layer to release the active ingredient into solution to pass through the perforations of the second water soluble control film to come into contact with the receiving site.

24. A method according to claim 23 including periodically re-wetting the first water soluble control film.

25. A method according to claim 23 wherein the perforations in the second water soluble control film are larger than the perforations in the first water soluble control film.
